(19) United States
(12) Patent Application Publication
(10) Pub. No.: US 2007/0141177 A1
(43) Pub. Date: Jun. 21, 2007

(54) Method for enhancing fertility

(76) Inventor: Max Rosenberg, Dallas, TX (US)

Correspondence Address: PILLSBURY WINTHROP SHAW PITTMAN, LLP, P.O. BOX 10500, MCLEAN, VA, 22102, US

(21) Appl. No.: 11/304,620

(22) Filed: Dec. 16, 2005

(51) Int. Cl. A61K 33/00

(52) U.S. Cl. 424/717

(57) ABSTRACT

The present invention provides a method for enhancing fertility in a human woman. The method comprises: providing a non-toxic composition containing an alkalizing agent; and administering said composition into a vagina of the woman at least 12 hours prior to her ovulation, such that a pH value of her vagina is raised temporarily above 7.0.

### FIELD OF THE INVENTION

The present invention relates to a method for enhancing fertility.

### BACKGROUND OF THE INVENTION

It is common that healthy females of reproductive age cannot conceive even though all known infertility tests appear normal. Such cases have their own diagnosis—“Unexplained Infertility.” The main method of determining whether an individual's case falls into the category of “Unexplained Infertility” is through a process of elimination that involves the individual undergoing a number of tests to rule out all other known causes of infertility.

It would be desirable to provide a method for enhancing fertility in individuals who are having difficulty conceiving even though all other known fertility tests appear normal.

### SUMMARY OF THE INVENTION

The present invention provides a method for enhancing fertility in a human woman. The method comprises: providing a non-toxic composition containing an alkalizing agent; and administering said composition into a vagina of the woman at least 12 hours prior to her ovulation, such that her vaginal pH value is raised temporarily above 7.0.

In another aspect of the invention, the method further comprises determining a time of ovulation prior to administering the composition.

In another aspect of the invention, the composition is administered into the vagina within a predetermined time period at least 12 hours prior to ovulation. The time period may be any suitable period, and may be, for example, within 7 days of ovulation, 2 to 7 days prior to ovulation, or 3 to 5 days before ovulation.

In another aspect of the invention, the composition is administered multiple times prior to ovulation.

In another aspect of the invention, the woman is diagnosed with unexplained infertility prior to administering the composition.

These and other features of the present invention and their attendant advantages will be more fully understood upon reading the following detailed description of the invention.

### DETAILED DESCRIPTION OF THE INVENTION

Candida is a genus of yeast-like fungi that is commonly part of the normal flora of the mouth, skin, intestinal tract and vagina of a human woman. Candida organisms require the element zinc for their sustained growth and survival. The inventor discovered that when vaginal Candida organisms come into contact with human semen, which has a high zinc content, the Candida may harm or damage the semen in an effort to acquire its zinc, thereby reducing its ability to progress to the cervix and into the uterus to fertilize the egg. Because Candida is normally present in the human vagina, this phenomenon may go undetected. In particular, the inventor discovered that Candida may reduce the semen's ability to fertilize when Candida exists at normal or slightly elevated levels, such that the Candida may not be diagnosed as an infertility problem. Assuming that there are no other identifiable reasons diagnosed for infertility, a woman may be diagnosed with “Unexplained Infertility.”

As such, the inventor has found that a potentially effective treatment method for this type of “Unexplained Infertility,” and thus an enhancement of fertility, includes temporarily disabling the biologic activity of Candida by raising the pH of the vagina at least 12 hours prior to ovulation. Candida albicans, the most common type of vaginal yeast, is most active at an acidic pH. Notably, a vaginal pH is around 3.0 to 4.5, a pH at which Candida albicans has a higher biologic, and thus zinc-acquiring, activity rate. The inventor believes that when the pH is raised to above 7.0, the Candida albicans' zinc-acquiring activity ceases, or is significantly reduced, temporarily without killing the Candida itself. The result is that the function of semen as a vehicle for sperm transport remains undamaged, thereby allowing the normal progression to fertilization, and yet the normal and desirable presence of Candida in the vagina is not disturbed.

The neutralization or suppression of Candida activity, and thus the enhancement of fertilization, is accomplished by the vaginal application of an alkalizing composition to temporarily raise the vaginal pH to above 7.0 prior to ovulation. A method for enhancing fertility includes determining the time of ovulation through any known method of calculation or estimation. Once this time has been determined, a non-toxic alkalizing composition is introduced vaginally at least 12 hours prior to ovulation.

It is understood that the determination of a woman's time of ovulation cannot be predicted perfectly. Thus, the invention contemplates that “determining” a woman's ovulation involves methods that approximate or estimate the time of ovulation. Any reference to a time of ovulation would be in reference to the time determined and not the actual time of ovulation (as that is not known until it occurs).

Prior studies have suggested douching with sodium bicarbonate immediately prior to intercourse in individuals with increased acidity or vaginitis in order to enhance fertility. It has been asserted that this douching neutralizes the acidity of the genital tract and removes most of the acidic vaginal discharge, thereby allowing an increased number of spermatozoa to be available to the cervical mucus. These studies rely upon an increase in vaginal pH to neutralize the acidity of the genital tract and rinse out most of the acidic vaginal discharge. McLeod et al., In Studies on Fertility, Edited by R. G. Hamsen, Oxford, Blackwell, 1958, at 41; Teague et al., Interference of human spermatozoa motility by Escherichia coli, Fertility and Sterility, (1971), at 281; Walters-Everhardt et al., Buffering capacity of human semen, Fertility and Sterility, (1986), at 114.

Other studies have emphasized that a benefit of introducing a sodium bicarbonate douche immediately prior to intercourse comes from the alkalizing of the cervical mucus. The increase in pH value, however, is only temporary, as the alkalizing effect does not last. This douching is performed just prior to intercourse, which may provide for less spontaneity in sexual interactions. It was originally believed that the cervical mucus did not play a role in infertility issues. It was assumed that cervical mucus is alkaline and remains unchanged throughout the menstrual cycle. However, modern studies have shown that specific conditions may lead to an acidification of the cervical mucus, which may lead to infertility in certain cases. Ansari, A. et al., Sodium Bicarbonate Douchingfor Improvement of the Postcoital Test, Fertility and Sterility, June 1980, at 608; Everhardt et al., Improvement of cervical mucus viscoelasticity and sperm penetration with sodium bicarbonate douching, Human Reproduction, (1990), at 133.

The present invention does not require that the vaginal pH value be raised immediately prior to intercourse. Once the Candida albicans' zinc-acquiring activity has been disrupted by the shock of the increased pH, the inventor believes that the disruption to the zinc-acquiring activity, although temporary, will effectively suppress the Candida's zinc-acquiring biologic activity through all, or at least a part of, the woman's ovulation period, and reduce its adverse impact on sperm.

The inventor believes that this suppression of Candida's biologic activity will continue even if the vaginal pH has increased prior to ovulation. Therefore, the alkalizing composition can be introduced up to 7 days prior to ovulation. Unlike the other studies, the alkalizing agent need not be introduced immediately prior to intercourse. Instead, the primary concern is whether the Candida albicans' zinc-acquiring activity has been sufficiently disabled prior to ovulation. Introduction immediately prior to intercourse, as taught by prior studies, is undesirable because there may be leftover byproducts in the vagina from the alkaline composition to interfere with the sperm's progression to the cervix. In contrast, by introducing the alkalizing agent in advance of ovulation, any such byproducts will likely be discharged by the time of ovulation.

The temporary disruption of the Candida albicans 'zinc-acquiring activity is beneficial because the presence of Candida in the vagina is normal and desirable, and eradicating the Candida would disrupt the normal biological balance of the vagina, leading to a host of other issues.

Although other non-toxic agents can be utilized, the preferred embodiment would utilize sodium bicarbonate as an alkalizing agent. The preferred embodiment is made up of a solution of water and 3.3% sodium bicarbonate. Sodium bicarbonate is the preferred agent because of its non-toxic properties, ready availability, rapid dissipation, water solubility and non-harmful effects on semen or sperm.

The composition containing the alkalizing agent may preferably have a pH in the range of 6.5 to 8.5, more preferably in the range of 6.0 to 8.0, and more preferably of 8.0. Although sodium bicarbonate is the preferred alkalizing agent, one of skill in the art would understand that numerous other alkalizing agents, such as other carbonates or phosphates, may also be employed.

The non-toxic alkalizing composition is introduced into the female's vagina within a predetermined time period prior to the determined time of ovulation. This predetermined time period may vary according to several variables, including the concentration of the non-toxic alkalizing agent and the number of times that the alkalizing composition is introduced, as the number of applications can vary between a single application to multiple applications. In one aspect of the invention, the non-toxic alkalizing composition is introduced after the female has been diagnosed with Unexplained Infertility.

The predetermined time period for introduction of the non-toxic alkalizing composition into the vagina at least 12 hours prior to ovulation. This time period may be 2 to 7 days prior to ovulation, 3 to 5 days prior to ovulation or anytime within 7 days prior to ovulation. As mentioned above, the non-toxic alkalizing composition may also be administered multiple times (2, 3 or more) during these time periods.

The preferred embodiment of this invention includes reaching the optimal pH by vaginally administering a liquid douche having an applicator head for administrating a 3.3% solution of sodium bicarbonate and water into the vagina 5 days prior to ovulation and then repeating the process 3 days prior to ovulation. It is believed that these two, spaced-apart treatments will effectively suppress the Candida's zinc-acquiring biologic activity through all, or at least a part of, the woman's ovulation period, and reduce its adverse impact on sperm. Likewise, it is believed that this will not adversely affect the viability of the vagina's Candida population.

Although a sodium bicarbonate solution is described as the preferred composition, one of skill in the art would understand that numerous other compositions, such as lotions, vaginal suppositories, medicated tampons, creams, jellies and gels, may also be employed. Also, although a sodium bicarbonate and water solution is described as the preferred composition, one of skill in the art would understand that numerous other additives, such as emulsifiers, perfumes and color additives, may also be employed.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

What is claimed is:

1. A method for enhancing fertility comprising:
providing a non-toxic composition containing a alkalizing agent; and
administering said composition into a vagina of the woman at least 12 hours prior to her ovulation, such that a pH value of her vagina is raised temporarily above 7.0.

2. The method according to claim 1, wherein the non-toxic alkalizing agent is sodium bicarbonate.

3. The method according to claim 2, wherein the composition is a solution comprising water and 3.3% sodium bicarbonate.

4. A method according to claim 1, further comprising:
determining a time of ovulation for the woman prior to administering the composition.

5. A method according to claim 4, wherein the composition is administered into the vagina within a predetermined time period prior to the determined time of ovulation.

6. The method according to claim 5, wherein the predetermined time period is 2 to 7 days.

7. The method according to claim 6, wherein the predetermined time period is 3 to 5 days.

8. The method according to claim 4, wherein the composition is administered into the vagina within 7 days prior to the determined time of ovulation.

9. A method according to claim 1, wherein the composition is administered into the vagina multiple times prior to the determined time of ovulation.

10. The method according to claim 9, wherein the alkalizing agent is sodium bicarbonate.

11. The method according to claim 10, wherein the composition is a solution comprising water and 3.3% sodium bicarbonate.

12. A method according to claim 9, further comprising:
determining a time of ovulation for the woman prior to administering the composition.

13. A method according to claim 12, wherein the composition is administered into the vagina within a predetermined time period prior to the determined time of ovulation.

14. The method according to claim 13, wherein the predetermined time period is 2 to 7 days.

15. The method according to claim 14, wherein the predetermined time period is 3 to 5 days.

16. The method according to claim 12, wherein the composition is first administered about 5 days prior to the determined time of ovulation and then about 3 days prior to the determined time of ovulation.

17. The method according to claim 4, wherein the composition disables a biologic activity of Candida present in the vagina.

18. The method according to claim 17, wherein the disabling of the biologic activity of the Candida is temporary.

19. The method according to claim 17, wherein a zinc-acquiring ability of the Candida is disabled.

20. The method according to claim 18, wherein a zinc-acquiring ability of the Candida is disabled temporarily.

21. The method according to claim 1, further comprising diagnosing the woman with unexplained infertility prior to administering the composition.

22. The method according to claim 4, further comprising diagnosing the woman with unexplained infertility prior to administering the composition.

23. The method according to claim 5, further comprising diagnosing the woman with unexplained infertility prior to administering the composition.

24. The method according to claim 6, further comprising diagnosing the woman with unexplained infertility prior to administering the composition.

25. The method according to claim 7, further comprising diagnosing the woman with unexplained infertility prior to administering the composition.

26. The method according to claim 9, further comprising diagnosing the woman with unexplained infertility prior to administering the composition.

27. The method according to claim 12, further comprising diagnosing the woman with unexplained infertility prior to administering the composition.

28. The method according to claim 13, further comprising diagnosing the woman with unexplained infertility prior to administering the composition.

29. The method according to claim 14, further comprising diagnosing the woman with unexplained infertility prior to administering the composition.

30. The method according to claim 15, further comprising diagnosing the woman with unexplained infertility prior to administering the composition.

31. The method according to claim 16, further comprising diagnosing the woman with unexplained infertility prior to administering the composition.

32. The method according to claim 17, further comprising diagnosing the woman with unexplained infertility prior to administering the composition.

33. The method according to claim 18, further comprising diagnosing the woman with unexplained infertility prior to administering the composition.

34. The method according to claim 19, further comprising diagnosing the woman with unexplained infertility prior to administering the composition.

35. The method according to claim 20, further comprising diagnosing the woman with unexplained infertility prior to administering the composition.

36. The method according to claim 1, wherein the composition is liquid.

37. The method according to claim 36, wherein the liquid is contained in a douche having an applicator head for enabling administration of the liquid into the vagina.

38. The method according to claim 1, wherein the composition is an aqueous gel.

39. A method for enhancing fertility comprising:
providing a non-toxic composition containing a alkalizing agent; and
administering said composition into a vagina of the woman having an initial pH at least a predetermined period of prior to her ovulation, such that a pH value of her vagina is raised temporarily from the initial pH to above 7.0;
wherein the predetermined period of time is selected such that (a) the pH value of her vagina returns to approximately to the initial pH prior to her ovulation and (b) a zinc acquiring ability of yeast in her vagina remains suppressed through at least part of her ovulation.
